(19) United States
(12) Patent Application Publication
(10) Pub. No.: US 2007/0015741 A1
(43) Pub. Date: Jan. 18, 2007

(54) Novel prodrugs of estradiol

(76) Inventors: James Keown, Kilkeel (IE); James William McIlroy, Belfast (IE); John Alexander King, Sallins (IE); Claire Gilligan, Belfast (IE); William Paul Armstrong, Belfast (IE)

Correspondence Address: FITZPATRICK CELLA HARPER & SCINTO, 30 ROCKEFELLER PLAZA, NEW YORK, NY, 10112, US

(21) Appl. No.: 11/478,584

(22) Filed: Jul. 3, 2006

Related U.S. Application Data
(60) Provisional application No. 60/698,866, filed on Jul. 12, 2005.

(51) Int. Cl. A61K 31/58; A61K 31/56; C07J 43/00

(52) U.S. Cl. 514/176; 514/182; 552/558; 540/107

(57) ABSTRACT

The present invention is a prodrug derivative of estradiol according to Formula I:

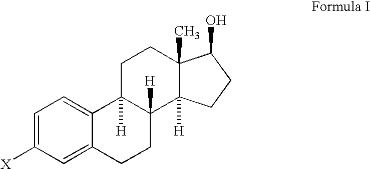

### CROSS REFERENCE TO RELATED APPLICATIONS

This application claims the benefit of U.S. Provisional Application Ser. No. 60/698,866 filed on Jul. 12, 2005.

### BACKGROUND OF THE INVENTION

1. Field of the Invention

This invention is directed to a prodrug derivative of estradiol and pharmaceutically acceptable salts thereof. The invention also includes pharmaceutical dosage units of the prodrug derivative.

2. Related Background Art

Unbound 17β-estradiol is the most active, naturally occurring human estrogen. However, due to poor absorption and extensive first-pass metabolism in the gastrointestinal tract and liver following oral absorption, it is not generally orally active. Several methods have been utilized to increase its oral activity. A micronized form (to provide an increased surface area of drug for absorption) of estradiol which has sufficient oral bioavailability to be active is available. Alternatively estradiol can be formulated as a conjugate, e.g., conjugated equine estrogens which are essentially estrogen metabolites purified from the urine of pregnant mares that contain sulfate and glucuronide derivatives (Martindale 32^{ed}, 1999, Pharmaceutical Press). These conjugates are orally active as they are hydrolyzed by enzymes in the lower gastrointestinal tract allowing absorption of the active estrogen. Another alternative is the oral administration of estradiol esters. Such compounds are known in the art for oral administration of estrogen and include estradiol-3,17-diacetate, estradiol-17-acetate, estradiol-3,17-valerate, estradiol-3-valerate and estradiol-17-valerate. These esters rapidly hydrolyze to free estradiol following oral administration.

U.S. Pat. No. 3,916,002 to Taubert et al. describes a number of oligomeric esters of androgenic, estrogenic and progestogenic steroids having the formula: R—O—CO—(CH₂)ₙ—CO—O—R, wherein n is between 2 and 8, and each R is a monovalent steroid radical. The steroid radical is derived from steroids having a hydroxyl substituent at one of the carbon atoms numbered 3, 16 or 17. They can be produced by esterification of the two carboxyl radicals of a dicarboxylic acid with a steroid alcohol having a hydroxyl radical substituent at carbon atoms numbered 3, 16, or 17.

A novel prodrug of estradiol that may increase oral activity would be highly advantageous.

### SUMMARY OF THE INVENTION

The present invention is a prodrug derivative of estradiol according to Formula I: 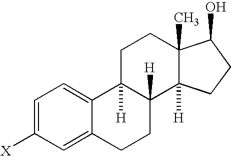
and enantiomers and pharmaceutically acceptable salts thereof, wherein X is selected from the group consisting of

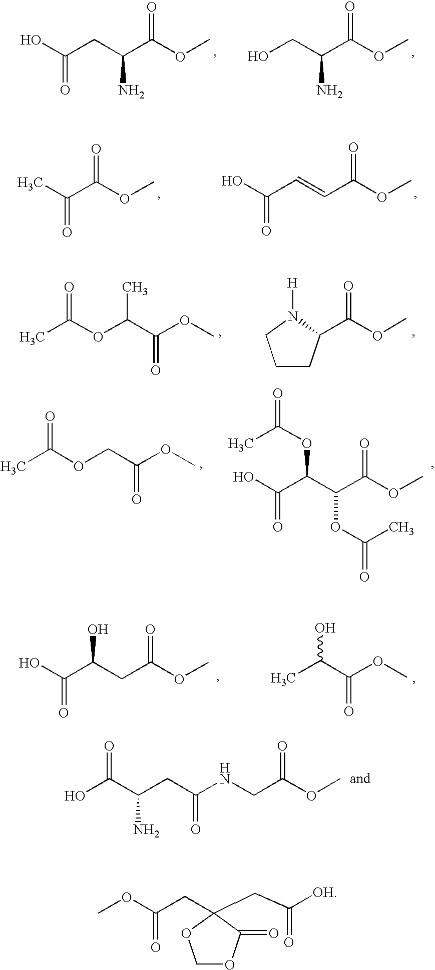

The present invention also includes a pharmaceutical dosage unit comprising (a) a prodrug of estradiol according to Formula I, and (b) one or more pharmaceutically acceptable excipients.

In another aspect of the present invention, a method of providing contraception is provided. The method comprises the step of administering to a patient in need thereof, an effective amount of a prodrug of estradiol of the invention, for an effective period of time.

In yet another aspect of the invention, a method of providing hormone treatment therapy is provided. The method comprises the step of administering to a patient in need thereof, an effective amount of a prodrug of estradiol of the invention, for an effective period of time.

### DETAILED DESCRIPTION OF THE INVENTION

For the purposes of the present invention, a prodrug is an entity which either comprises an inactive form of an active drug or includes a chemical group which confers preferred characteristics on the drug.

For the purposes of the present invention, room temperature is understood to mean 25° C.+/−5° C.

In the present invention, the prodrugs of estradiol have an X group attached to at the 3° C. position of an estradiol moiety. It should be understood that the inventive compounds of Formula I include their enantiomers and their pharmaceutically acceptable salts.

The prodrug of estradiol of the invention has the structural formula:

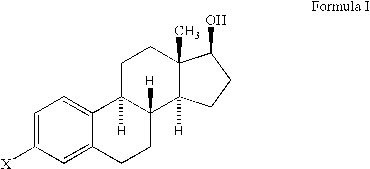

wherein X is selected from the group consisting of

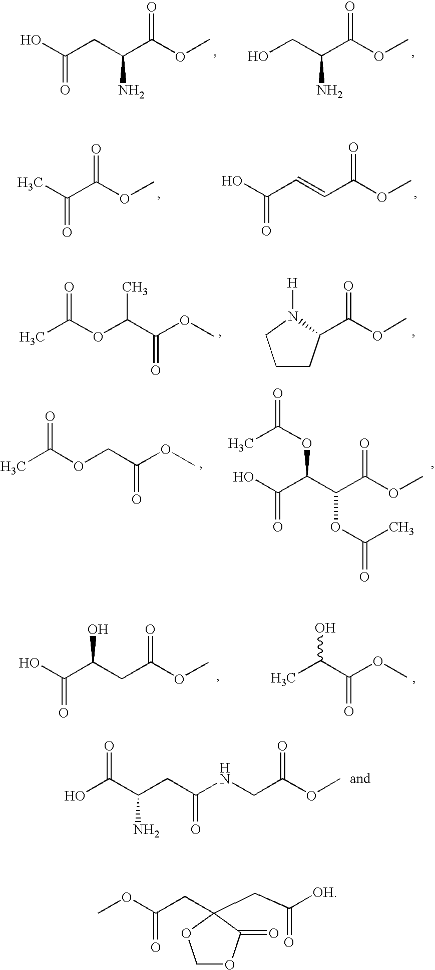

In a preferred embodiment, X is selected from the group consisting of:

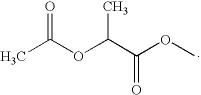

Notably, X attaches to the estradiol compound at the 3′C position of the estradiol compound. It should be understood that the inventive compounds of Formula I include all their enantiomers and their pharmaceutically acceptable salts.

As used herein, the phrase “pharmaceutically acceptable salt” refers to a salt that retains the biological effectiveness of the free acids and bases of a specified compound and that is not biologically or otherwise undesirable.

A pharmaceutical dosage unit may be formulated to include the prodrug derivative of estradiol of the present invention in combination with one or more pharmaceutically acceptable excipients.

Excipients useful herein include a wide variety of additives, or ingredients, such as for example, fillers, diluents (solid and liquid), biocompatible polymers (such as organopolysiloxanes, polyurethanes and polymethylacrylates), skin penetrators and penetration enhancers, solubilizers, lubricants, stabilizers, flow control agents, colorants, glidants, effervescent agents, sweeteners, flavors, perfumes, and the like.

Other steroids, e.g., progestogens may be included in the pharmaceutical dosage unit. Exemplary progestogens include norethindrone, drospirenone, trimegestone, levonorgestrel, desogestrel, 3-ketodesogestrel, gestodene, demegestone, dydrogesterone, medrogestone, medroxy progesterone and esters thereof and the like.

The pharmaceutical dosage unit may be in an orally ingestible form, such as tablets, capsules, chewable tablets or capsules, troches, liquid suspensions, pills, or sustained release dosage forms. Alternatively, the pharmaceutical dosage unit may be a transdermal delivery system. Or in another embodiment the pharmaceutical dosage unit may be a topical composition such as a gel, cream, ointment, liquid and the like. Or in an alternative embodiment, the pharmaceutical dosage unit may be designed for vaginal administration e.g., a vaginal ring.

The prodrug derivatives of estradiol may be synthesized using the methods described herein. These methods may be modified or alternative synthesis methods may be employed as desired. The synthesis methods typically begin with estradiol as the starting material. It should be understood, however, that where estradiol is indicated, derivatives of estradiol may be used.

In one embodiment, a fumaric acid estradiol ester may be formed in accordance to Reaction Sequence 1. The reaction combines estradiol and maleic anhydride in the presence of a base catalyst, e.g., sodium hexamethyldisilylamide (NaHMDS) and a solvent, e.g., tetrahydrofuran (THF) at −78° C. Deprotecting agents such as hydrochloric acid (HCl) and ether are then added to yield the desired product.

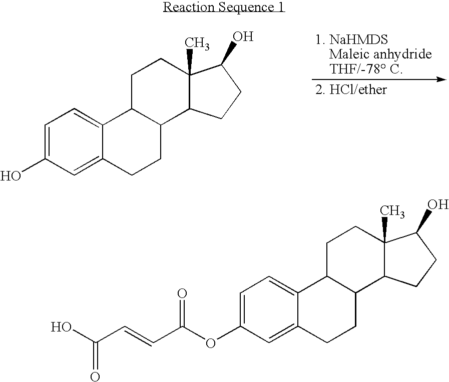

Reaction Sequence 2 provides a route to synthesize a derivative estradiol ester compound by reacting estradiol or a derivative thereof with a compound having the structure 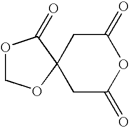
in the presence of NaHMDS, maleic anhydride, and THF (−78° C.) to form an intermediate compound, which is then reacted with HCl/dioxane.

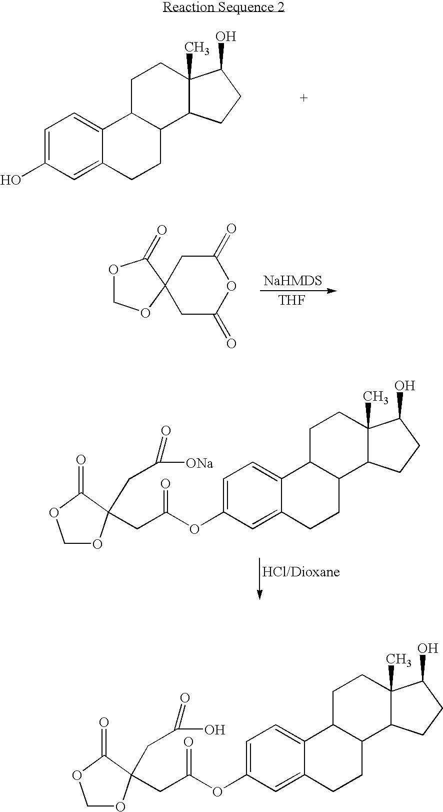

In another embodiment, a prodrug compound of the invention may be synthesized by reacting estradiol directly with a compound having a structure

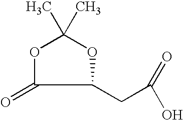

The intermediate compound undergoes deprotection and forms a malic acid estradiol ester, as depicted in Reaction Sequence 3. 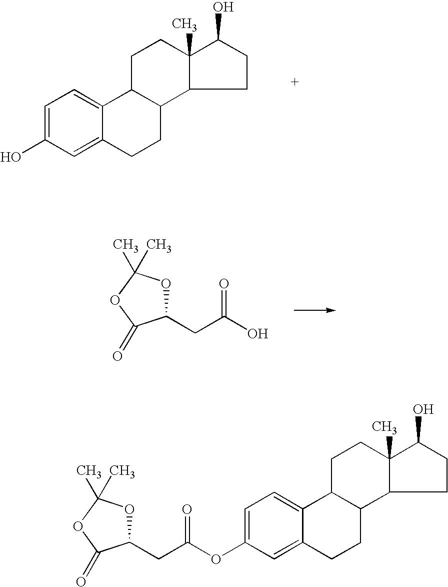
Followed by deprotection step

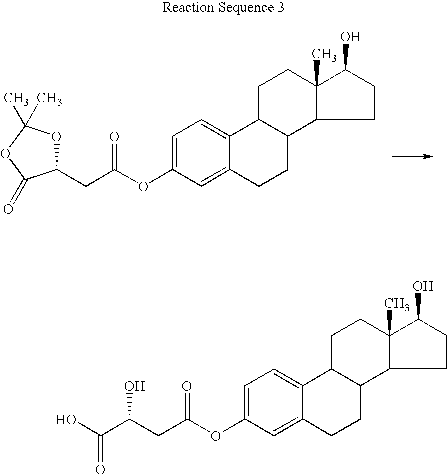

In Reaction Sequence 4, estradiol is reacted with pyruvic acid. An intermediate compound is formed, which is then treated with a deprotecting agent, such as sodium borohydride (NaBH₄). The resulting compound is the lactic acid estradiol ester.

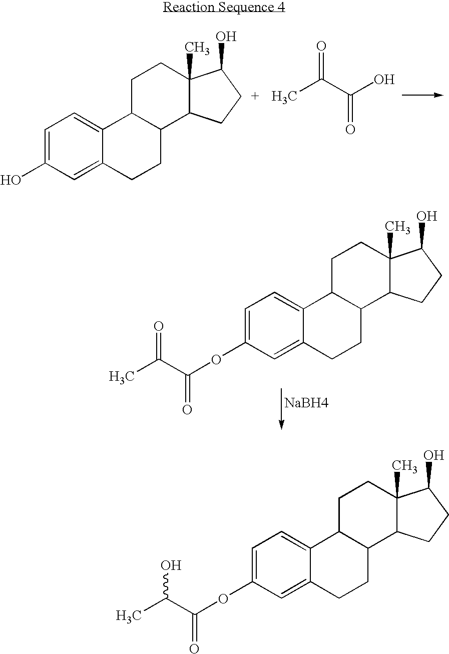

In Reaction Sequence 5, an acetoxyacetic acid ester of estradiol is synthesized by reacting estradiol with acetoxyacetic acid.

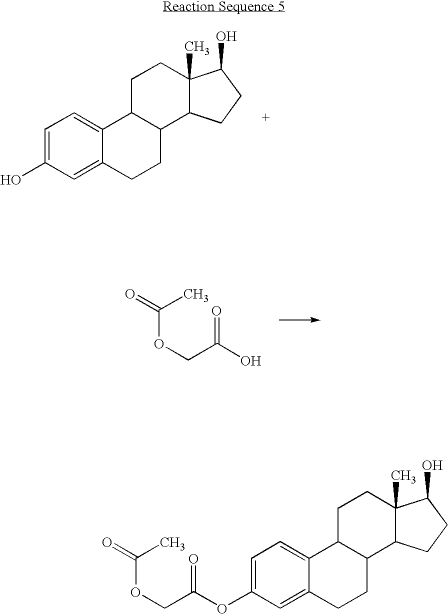

A prolinate estradiol ester derivative may be formed in accordance to Reaction Sequence 6. Estradiol is combined with Boc-proline in the presence of a coupling agent, e.g., DCC, forming an intermediate compound. A deprotecting agent, such as HCl/dioxane, is then added to form the desired prolinate estradiol ester derivative.

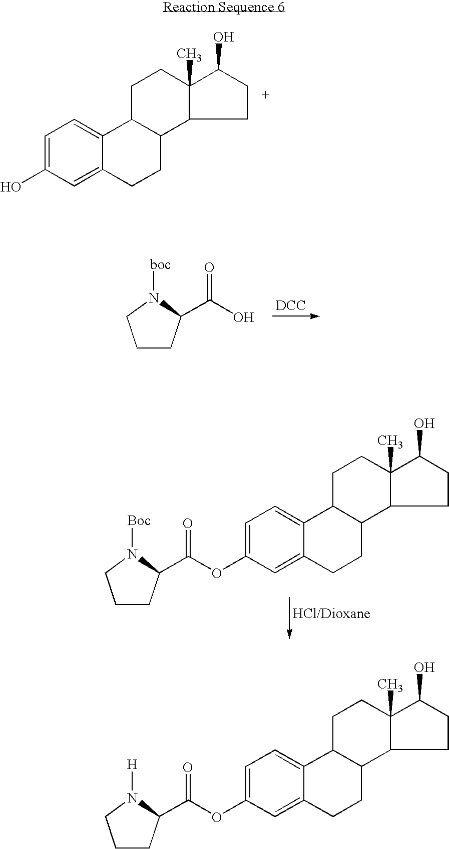

Reaction Sequence 7 provides a synthesis route for making a serine estradiol ester derivative. Estradiol is combined with Boc-serine. An intermediate compound is formed which is then reacted in the presence of a deprotecting agent, such as HCl/dioxane, to produce the serine estradiol ester.

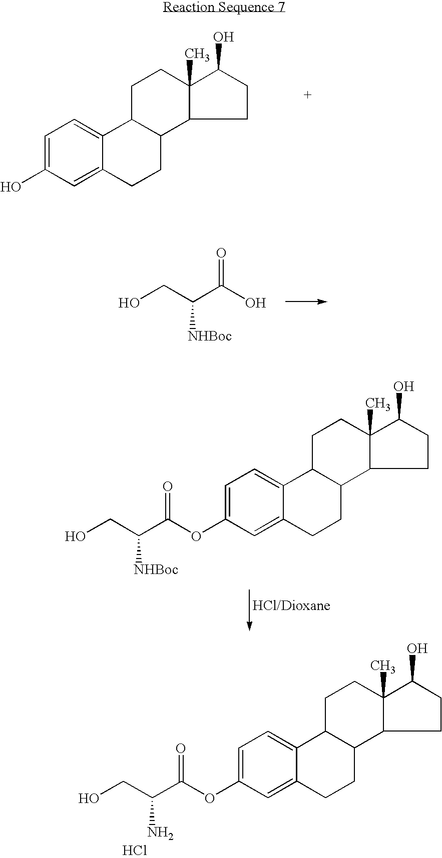

Reaction Sequence 8 provides a synthesis route for making the acetyl lactic acid ester derivative of estradiol, i.e., estradiol lactate-acetate. Estradiol is combined with acetyl lactic acid to form the desired compound.

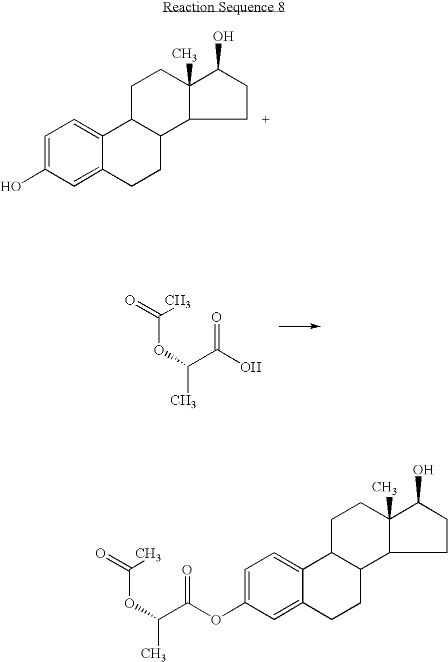

Utilizing Reaction Sequence 9, estradiol is combined with a compound having the structure 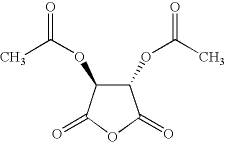
to form an intermediate compound that is then treated with a deprotecting agent, such as HCl/ether, to yield diacetyltartaric acid estradiol ester.

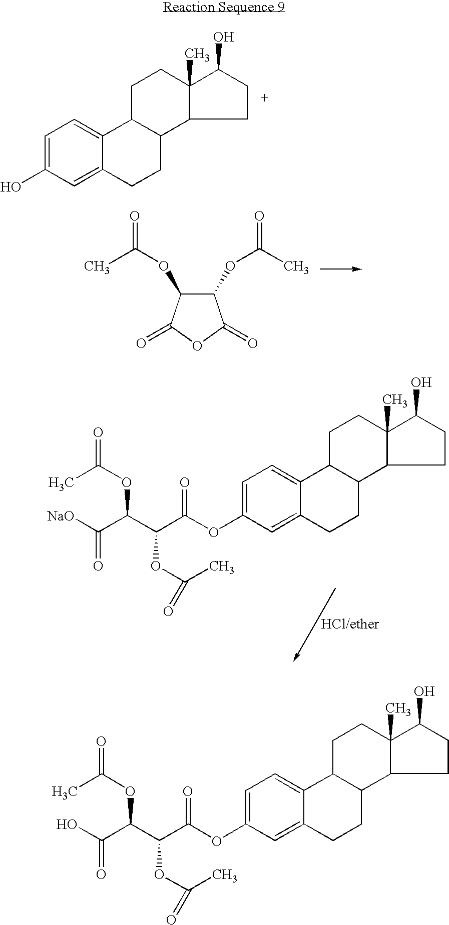

Reaction Sequence 10 depicts a process for synthesizing an Asp-Gly estradiol ester. Estradiol is combined with Boc-amino acetic acid, which forms an intermediate compound. As shown in Reaction Sequence 10, a compound having the structure 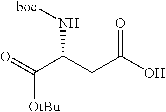
is added to the intermediate compound, which is then treated with HCl to form the desired prodrug estradiol derivative ester.

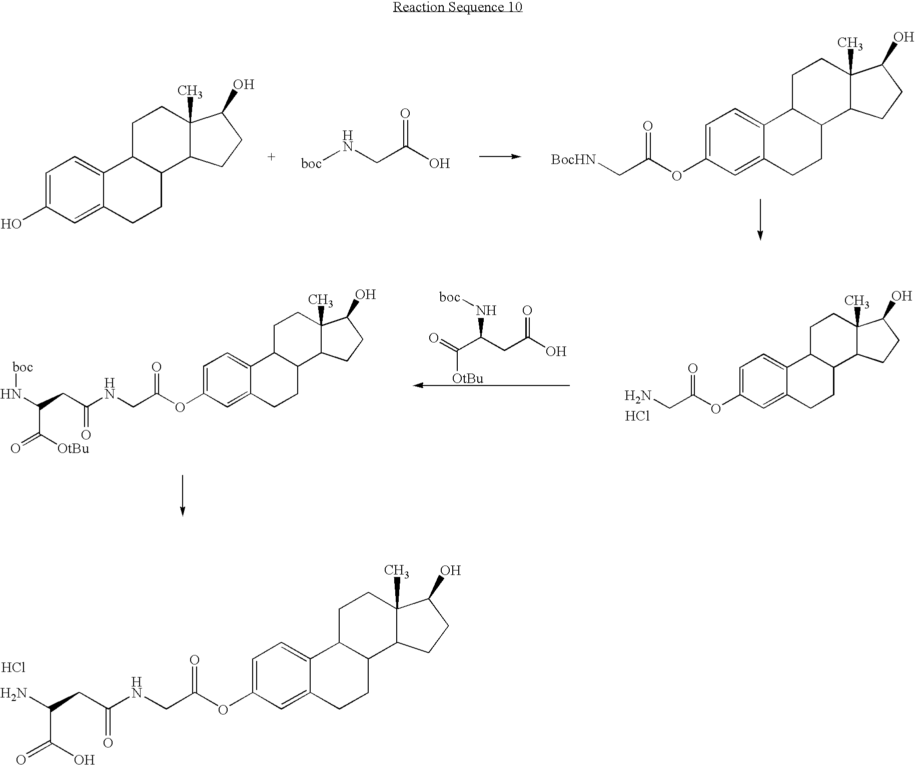

Reaction Sequence 11 starts by combining estradiol with Boc-aspartic acid t-butyl C₄. This combination forms an intermediate compound, which is then treated with a deprotecting agent, such as HCl/dioxane to yield the aspartic acid estradiol ester.

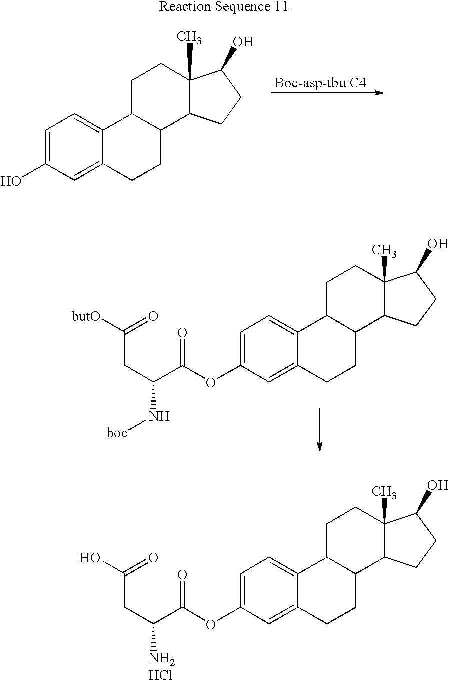

Utilizing Reaction Sequence 12, estradiol is reacted with Boc-aspartic acid t-butyl C₁, which forms an intermediate compound. A deprotecting agent, such as HCl/dioxane is combined with the intermediate compound to form the desired aspartic acid estradiol ester.

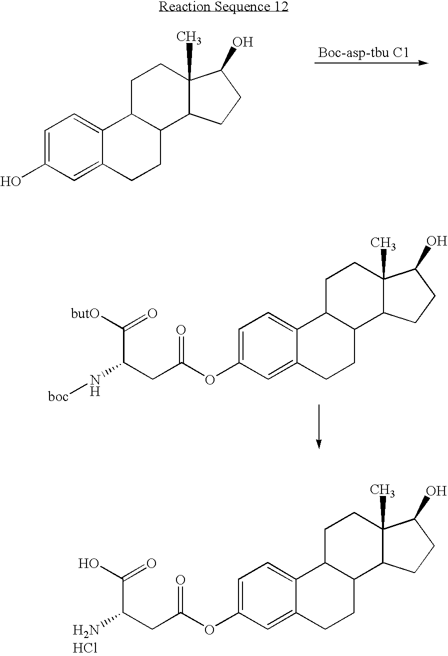

As noted above, where estradiol is indicated, derivatives of estradiol may be used.

Coupling agents that may be used in synthesizing the prodrug derivative of ethinyl estradiol of the present invention, may be for example, bis(4-nitrophenyl)carbonate (b-NPC), N,N′-dicyclohexyl-carbodiimide (DCC), 1-(3-dimethylaminopropyl)-3-ethyl-carbodiimide hydrochloride (EDCI), and mixtures thereof. Alternative compounds may be used, so long as they fulfill the intended purpose.

Deprotecting agents may be used in the synthesis reactions when needed. Non-limiting examples include HCl, dioxane, ether, sodium borohydride (NaBH₄), and mixtures thereof such as, for example, acetic acid:THF:water.

In the synthesis reactions described, a base may be used as a catalyst. Suitable bases include, but are not limited to DMAP, triethylamine, NaHMDS or mixtures thereof.

Solvents that may be used in the synthesis reactions are for example, tetrahydrofuran (THF), chloroform, dichloromethane, and the like. However, it should be understood that many other organic solvents may be suitable.

To increase the purity of the prodrug of estradiol, the prodrug may be treated to one or more washing steps, and/or recrystallization steps.

The washing step may be used to rinse the precipitate that is formed by the prodrug of estradiol. As noted, one or more washing steps may be used. Water, sodium hydroxide, or any suitable alternative can be generally used for washing purposes.

As previously noted, the purity may be increased by subjecting the prodrug to one or more recrystallization steps. The recrystallization step may be performed by various methods, and using suitable solvents such as but not limited to ethyl acetate, hexane or THF, or mixtures thereof.

The drying step in the synthesis may be conducted by various methods including but not limited to, air drying, vacuum drying, oven drying, filtration, and the like. Drying may be enhanced by using a drying agent such as magnesium sulfate to assist in drying the product.

The prodrug of estradiol compounds of the present invention have been characterized using various analytical methods. For example, high performance liquid chromatography (HPLC) was used to establish the purity of the synthesized product. ¹H and ¹³C nuclear magnetic resonance (NMR), mass spectrometry and infrared (IR) spectroscopy were used to verify its structure. Moreover, the product was further characterized by determining the melting point.

The prodrugs of the invention may be used for providing contraception. A therapeutically effective amount of the prodrug of estradiol of the invention is administered to a patient in need thereof, for an effective period of time. Preferably, the prodrug is administered in combination with a progestogen.

The prodrug of estradiol of the invention can also be used in providing hormone treatment therapy. Such a method of treatment would comprise the step of administering to a patient in need thereof, a therapeutically effective amount of a prodrug of estradiol of the invention, for an effective period of time.

The prodrugs of estradiol of the present invention are administered in a “therapeutically effective amount.” This is understood to mean a sufficient amount of a compound or composition that will positively modify the symptoms and/or condition to be treated. The therapeutically effective amount can be readily determined by those of ordinary skill in the art, but of course will depend upon several factors. For example, one should consider the condition and severity of the condition being treated, the age, body weight, general health, diet, and physical condition of the patient being treated, the duration of the treatment, the nature of concurrent therapy, the particular active ingredient being employed, the particular pharmaceutically-acceptable excipients utilized, the time of administration, method of administration, rate of excretion, drug combination, and any other relevant factors.

The prodrugs of the invention are preferably administered orally, transdermally, topically or vaginally. The preferred dosage forms are tablets, semi-solid dosage forms such as creams or gels, or vaginal rings.

Specific embodiments of the invention will now be demonstrated by reference to the following examples. It should be understood that these examples are disclosed solely by way of illustrating the invention and should not be taken in any way to limit the scope of the present invention.

### EXAMPLES

The following is an example of stability for the preferred compound described previously. Stability was conducted at 40° C./75% RH, with the prodrug being assayed at specific time-points for degradation to the parent compound, estradiol. The time-points for stability were T=0 months, T=2 weeks, T=1 month, and T=3 months.

The results of these studies on estradiol lactate-acetate, which may be prepared according to reaction sequence 8, are shown in TABLE 1.

TABLE 1

| | | | Assay | (%) |
|---|---|---|---|---|
| Prodrug Monomer | T = 0 | T = 2 W | T = 1 | T = 3 |
| Estradiol Lactate-Acetate | 94.45 | 93.47 | 92.77 | 94.3 |

The following outlines the conditions utilized for analysis of the prodrug. Analysis was conducted using High-Performance Liquid Chromatography (HPLC). The retention time for estradiol lactate acetate was approximately 9.0 minutes using these conditions.

Conditions for HPLC analysis:

| | |
|---|---|
| Column: | Symmetry Shield RP₁₈ 5 um, 4.6 × 250 mm |
| Flow rate: | 1.0 mL/min |
| Temperature: | Ambient |
| Wavelength: | 210 nm |
| Injection Volume | 10 μL |
| Sample solvent: | MeCN (acetonitrile) |
| Retention Time: | ˜9.0 minutes |

As can be seen from TABLE 1, the estradiol lactate acetate was assayed at 94.3% after storage at 3 months at 40° C./75% RH.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

What is claimed is:

1. A prodrug derivative of estradiol having the following formula: 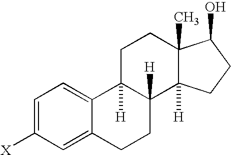
and enantiomers and pharmaceutically acceptable salts thereof, wherein X is selected from the group consisting of

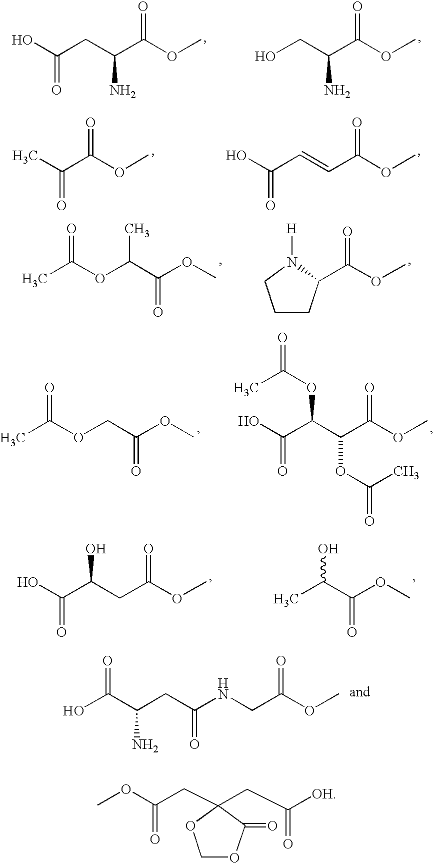

2. The prodrug of claim 1, wherein X is

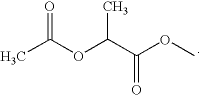

3. A pharmaceutical dosage unit comprising:
(a) a prodrug derivative of estradiol having the following formula: 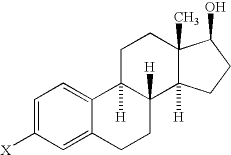
and enantiomers and pharmaceutically acceptable salts thereof, wherein X is selected from the group consisting of 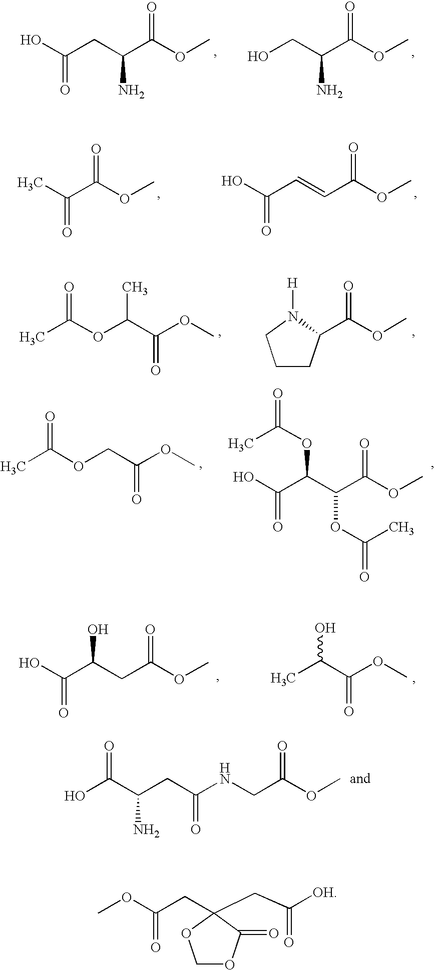
and
(b) one or more pharmaceutically acceptable excipients.

4. A method of providing contraception comprising the step of:
administering to a patient in need thereof, an effective amount of said prodrug derivative of estradiol of claim 1, for an effective period of time.

5. A method of providing hormone treatment therapy to a patient in need thereof, comprising the step of:
administering to said patient in need thereof, an effective amount of said prodrug derivative of estradiol of claim 1, for an effective period of time.
